(19) United States
(12) Patent Application Publication
(10) Pub. No.: US 2021/0378883 A1
(43) Pub. Date: Dec. 9, 2021

(54) FEMININE SANITARY FOLD DEVICE

(71) Applicant: LYNNEA LANG, GULFPORT, MS (US)

(72) Inventor: LYNNEA LANG, GULFPORT, MS (US)

(21) Appl. No.: 17/342,538

(22) Filed: Jun. 8, 2021

Related U.S. Application Data
(60) Provisional application No. 63/036,400, filed on Jun. 8, 2020.

(51) Int. Cl. A61F 13/472

(52) U.S. Cl. CPC A61F 13/47227; A61F 2013/4562; A61F 13/47236

(57) ABSTRACT

A feminine sanitary fold device that is constructed from a soft, flexible, and absorbent material is disclosed herein. The device can be used to absorb fluids related to menstruation in a sanitary and hygienic manner. The feminine sanitary fold device is composed of absorbent organic materials, and free of chemicals. The feminine sanitary fold device is distinctly shaped, generally conforming to the shape of a woman's vagina. In particular, the feminine sanitary fold is structured as a “fold” of material that is generally curved inward. Thus, the shape of the device is particularly adapted to allow the “fold” to be form-fitted to the inward curve of a woman's anatomy and rest comfortably at the vaginal opening, between the folds of vagina for secure and stable positioning during use.

### FIELD OF DISCLOSURE

The present disclosure generally relates to a product that can be used for feminine hygiene, particularly to absorb fluid related to feminine discharge, such as menstruation, in a sanitary and hygienic manner.

### BACKGROUND OF THE DISCLOSURE

In general, the types of feminine hygiene products that are available to women for providing protection and safety during menstruation is extremely limited. Currently, these products are dominated in the market by sanitary napkins (or pads) and tampons. However, there are number of issues that many women experience when using sanitary napkins and tampons. For example, sanitary napkins are more prone to leakage because they are typically applied to underwear, which results in gaps of unprotected space between the area where fluid collects and the absorbent material of the sanitary napkin. Also, because sanitary napkins are attached to underwear using a slight adhesive, they often shift from their initial position as a woman moves (e.g., exercise, physical activity). This shifting is undesirable, and increases the likelihood that the absorbent portion of the sanitary napkin is no longer optimally positioned to prevent leaks. Tampons generally can provide better protection than sanitary napkins. However, because tampons are inserted (as opposed to being attached to a garment), they can be uncomfortable while being worn, and have been linked with causing internal infections (e.g., toxic shock syndrome) in women. Therefore, it may be desirable to provide a feminine hygiene product that is an improvement over both sanitary napkins and tampons by providing enhanced protection, while being comfortable and safe for women to wear.

### BRIEF OVERVIEW

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are and explanatory only and are not restrictive of the disclosure, as claimed.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 depicts an example of a feminine sanitary fold device, in accordance with an exemplary embodiment of the present disclosure.

FIG. 2 depicts a top view of the feminine sanitary fold device shown in FIG. 1, in accordance with an exemplary embodiment of the present disclosure.

FIG. 3 depicts a front view of the feminine sanitary fold device shown in FIG. 1, in accordance with an exemplary embodiment of the present disclosure.

FIG. 4 depicts another front view of the feminine sanitary fold device shown in FIG. 1, in accordance with an exemplary embodiment of the present disclosure.

FIG. 5 depicts a bottom view of the feminine sanitary fold device shown in FIG. 1, in accordance with an exemplary embodiment of the present disclosure.

FIG. 6 depicts an exploded view of the feminine sanitary fold device shown in FIG. 1 illustrating the various layers comprising the multi-layer material, in accordance with an exemplary embodiment of the present disclosure.

FIG. 7 depicts another exploded view of the feminine sanitary fold device shown in FIG. 1 illustrating the various layers comprising the multi-layer material, in accordance with an exemplary embodiment of the present disclosure.

FIG. 8 depicts a cut-away view of the feminine sanitary fold device shown in FIG. 1 illustrating the various layers comprising the multi-layer material, in accordance with an exemplary embodiment of the present disclosure.

FIG. 9 is an example of a method for installing the sanitary fold device shown in FIG. 1 by a wearer, in accordance with an exemplary embodiment of the present disclosure.

FIG. 10 is an example of a method for removing the sanitary fold device shown in FIG. 1 by a wearer, in accordance with an exemplary embodiment of the present disclosure.

FIG. 11 depicts an example of the feminine sanitary fold device shown in FIG. 1 positioned to be installed by a wearer, in accordance with an exemplary embodiment of the present disclosure.

FIG. 12 depicts an example of the feminine sanitary fold device shown in FIG. 1 positioned while inserted in the wearer after being installed, in accordance with an exemplary embodiment of the present disclosure.

FIG. 13 depicts an example of a user in an initial standing position and the position of the feminine sanitary fold device shown in FIG. 1 in order to be installed, in accordance with an exemplary embodiment of the present disclosure.

FIG. 14 depicts an example of a user in a squatting position and the position of the feminine sanitary fold device shown in FIG. 1 while installing, in accordance with an exemplary embodiment of the present disclosure.

FIG. 15 depicts an example of a user in a squatting position and the feminine sanitary fold device shown in FIG. 1 in a partially inserted position while installing, in accordance with an exemplary embodiment of the present disclosure.

FIG. 16 depicts an example of a user in a squatting position and the feminine sanitary fold device shown in FIG. 1 in a fully inserted position while installing, in accordance with an exemplary embodiment of the present disclosure.

FIG. 17 depicts an example of a user in a standing position and the feminine sanitary fold device shown in FIG. 1 in the fully inserted position after installing, in accordance with an exemplary embodiment of the present disclosure.

FIG. 18 depicts an example of a user in a partially squatting position and the feminine sanitary fold device shown in FIG. 1 in the fully inserted position prior to removing, in accordance with an exemplary embodiment of the present disclosure.

FIG. 19 depicts an example of a user in a fully squatting position and the feminine sanitary fold device shown in FIG. 1 in a partially inserted position while removing, in accordance with an exemplary embodiment of the present disclosure.

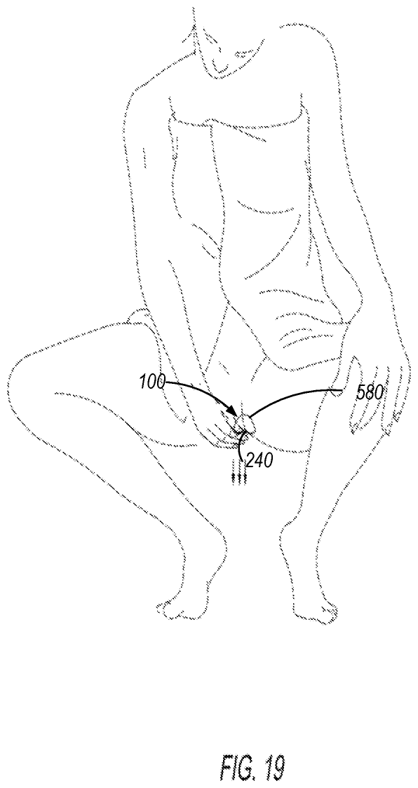

FIG. 20 depicts an example of a user in a standing position and the feminine sanitary fold device shown in FIG. 1 in a fully removed position after removing, in accordance with an exemplary embodiment of the present disclosure.

FIG. 21 depicts another example of a user in the squatting position and the feminine sanitary fold device shown in FIG. 1 in the fully inserted position, in accordance with an exemplary embodiment of the present disclosure.

### DETAILED DESCRIPTION OF THE DISCLOSURE

The present disclosure includes many aspects and features. Moreover, while many aspects and features relate to, and are described in, the context of a feminine sanitary fold device, embodiments of the present disclosure are not limited to use only in this context. The present disclosure can be understood more readily by reference to the following detailed description of the disclosure and the Examples included therein.

Before the present articles, systems, apparatuses, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Herein, regarding the following term, biodegradability, it may refer to a substance that may be decomposed into gas such as carbon dioxide, methane, water, and/or biomass under anaerobic or aerobic conditions according to the natural process under the existence of bacteria represented by actinomycetes and other microbes, and also means that the biodegradability (biodegradable rate and biodegradable degree) of the Substance equals to a material naturally generated such as fallen leaves or a synthetic polymer generally recognized having the same biodegradability under the same environment. Herein, regarding the following term, water dispersibility, it may mean the same as water degradability, where there is no effect from the limited amount of water (menstrual blood) upon use, whereas in conditions of large amounts of water or under water flow, the fibers are easily dispersible into at least small pieces which cannot clog the toilet plumbing. Herein, regarding the following term, water solubility, it may mean the property of not being affected by limited amount of water (menstrual blood) upon use, but being soluble in large amounts of water or under a flow of water.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed apparatuses, systems, and articles of the disclosure as well as the apparatuses themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and apparatuses of the disclosure. Thus, if there are a variety of additional steps that can be performed it is understood that each of these additional steps can be performed with any specific aspect or combination of aspects of the methods of the disclosure.

It is understood that the apparatuses and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

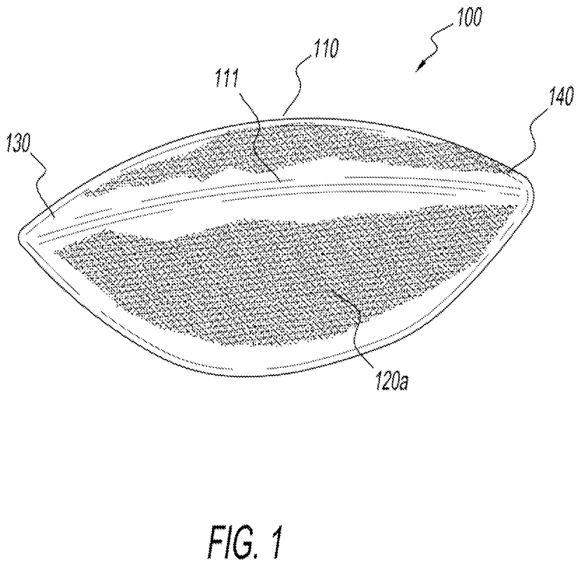

With reference now to the drawings, and in particular FIG. 1 through FIG. 7 thereof, examples of the feminine sanitary fold device and the principles and concepts thereof will be described.

Consistent with embodiments of the present disclosure, disclosed herein is a feminine sanitary fold device. As a general description, the feminine sanitary fold device is constructed from a soft, flexible, and absorbent material that can be used to absorb fluids related to feminine discharge (e.g., blood during a menstruation period, or urine from incontinence) in a sanitary and hygienic manner. The feminine sanitary fold device is composed of absorbent organic materials, and free of chemicals which allows the device to be safe and sanitary even when applied to a women's sensitive areas. The feminine sanitary fold device is distinctly shaped, generally conforming to the shape of a woman's vagina. In particular, the feminine sanitary fold is structured as a “fold” of material that is curved inward. Thus, the shape of the device is particularly adapted to allow the “fold” to be form-fitted to the inward curve of a woman's anatomy and rest comfortably at the opening of the vagina, i.e. vaginal opening between the folds of the labia majora and labia minora.

Once a wearer has properly applied the feminine sanitary fold device, fluid (e.g., menstruation) can be collected directly from the vaginal opening. Due to the feminine sanitary fold device conforming to the internal curves of the vagina, there is no empty space between the vaginal opening (where fluid exits) and absorbent material. Thus, the feminine sanitary fold device can improve protection over sanitary napkins that commonly have this area of unprotected space. Accordingly, the feminine sanitary fold device is distinctly designed to greatly reduce fluid leakage that may be typically experienced by women when using conventional feminine hygiene products, such as tampons, sanitary napkins, feminine pads, or cups that are not form fitted to a woman's anatomy.

Furthermore, the feminine sanitary fold device is not invasive. For example, the feminine sanitary fold device is primarily positioned at the vaginal opening (e.g., resting between the folds of the labia majora and labia minora) as compared to tampons that are inserted inside of the vagina (e.g., up to the cervix). Since the feminine sanitary fold device does not need to be inserted deeply inside of the vagina, there is no discomfort or risk of infection that is often related to tampon use. In some embodiments, the feminine sanitary fold device comes in a variety of sizes as options for optimally fitting the anatomy of various different women (having distinct sizes and shapes). Also, the feminine sanitary fold device can come in various flesh-toned colors to be worn more discreetly (e.g., matching underwear color or skin tone color). Due to the unique design of the feminine sanitary fold device, a similarly distinct method of using the same is also disclosed herein. The feminine sanitary fold device can be applied by the wearer squatting, and then manually placing the device at the vaginal opening between the folds of the labia majora and labia minora. There is no need for adhesive because, as the wearer stands, the folds of the labia majora and labia minora place slight pressure on the device keeping it in place. Additionally, the wearer can put on underwear (or other clothing) over the device to provides extra security. In order to remove the feminine sanitary fold device, the wearer can squat, releasing the pressure that is on the device keeping it in place. Then, the wearer can easily remove the device from the vaginal opening, by hand. In some embodiments, the feminine sanitary fold device is made of a biodegradable and flushable material, which makes removing and disposing of the device even more convenient when the wearer is in a bathroom. Also, after use, the wearer can remove a soiled feminine sanitary fold device to replace it with an unused device for continued protection.

Referring now to FIG. 1, an example of a feminine sanitary fold device 100 is shown. In the illustrated example, the device 100 is shown to have different portions (or areas), including: the curved portion 110; the front fold portion 130; the rear fold portion 140; and a lateral side portion 120a. It should be appreciated that FIG. 1 depicts a perspective view of the device 100, where lateral side portion 120a is prominently shown but an opposing lateral side is not visible. However, the “fold” shape of the device 100 involves two lateral sides that extend downward from the curved portion 110 of the device 100. In an embodiment, the curved portion 110 can comprise a portion of absorbent material 111. As alluded to above, the feminine sanitary fold device 100 is designed to have a distinct “fold” shape, having a curved portion 110 that is an inward concaved curve which generally mimics the inwardly curved form of a woman's vagina. Thus, the curved portion 110 is intended to fit inside of the inward curve of the vagina having its bend proximal (slight in contact with) the vaginal opening. In other words, the curved portion 110 is to be placed at the vaginal opening when worn. Further, when the device 100 is worn, the lateral side portion 120a can press against a corresponding side of the vagina. For instance, the lateral side portion 120a contacts the folds of the labia majora and labia minora on the right side of the vagina, while the opposing lateral side portion (not shown) contacts the opposite left side of the vagina. As the lateral side portion 120a contacts the folds of the labia majora and labia minora contact friction holds (associated with skin touching material) the device 100 into place. Even further, as the wears closes their legs (e.g., stands) which generally puts closing pressure in the vagina area, the labia majora and labia minora place pressure onto the lateral side portion, such as portion 120a, that it is in contact with.

As seen, the feminine sanitary fold device 100 has a length that extends from the front fold portion 130 to the rear fold portion 140. In an embodiment, the length of the device 100 is such that it can cover the entire length of vaginal opening. Additionally, the device 100 can have a width that extends from the lateral side portion 120a to the opposing lateral side portion (now shown). The width of the device 100 allows for it to be stably positioned for wear, by resting between the folds of the labia majora and labia minora. Also, the device 100 is constructed from a flexible material, which allows the device 100 to slightly bend and contract (or expand) to better improve its ability to form-fit to specific dimensions. For example, the wearer can slightly pinch the device 100 by hand, causing the lateral side 120a to be closer to the other lateral side in order to fit a more narrow opening. Consequently, the feminine sanitary fold device 100 is optimally shaped to fit the wearer in a manner that allows the device 100 to be stably positioned without unintended shifting, the use of adhesives, or being inserted deep into the vagina.

As previously described, a portion of absorbent material 111 can be integrated into a top surface of the curve (e.g., apex) of the curved portion 110. Thus, when worn, it is the absorbent portion 111 of the “fold” that comes into contact with the vaginal opening where fluid collects and exits the body. Therefore, the feminine sanitary fold device 100 has a shape that can improve leakage protection, by placing the absorbent material 111 in a position to best absorb fluid at its exit point (e.g., no large gap of unprotected space). Women can wear the feminine sanitary fold device 100 having confidence in its stability and protection, thereby not worrying about stains in clothing, and shifting during physical activity (e.g., exercise) that often occur with conventional feminine hygiene products.

Furthermore, the feminine sanitary fold device 100 can have a front fold portion 130 that is shaped to be substantially similar to the anatomy of the front of the vagina (e.g., facing the stomach). Similarly, the rear fold portion 140 of the device 100 can be shaped to be substantially similar to the anatomy of the rear of the vagina (e.g., facing the buttocks). Thus, the device 100 has an overall geometry that is form-fitting to a vagina, such that is can improve secure positioning, stability, and comfortable fit.

The feminine sanitary fold device 100 can be entirely constructed from a lightweight, flexible, and absorbent material, such as organic bamboo, absorbent cotton, and the like. As a result, the absorbent material can absorb menstrual fluid at any point of contact with the device 100. Alternatively, the device 100 may be constructed from the other non-absorbent material, and having the absorbent material only in the absorbent material 111 portion. As an example, the absorbent material 111 portion can be a strip of absorbent cotton that covers an area at the center of the curved portion 110. Further, using organic material to construct the device 100, such as bamboo, will also allow the device to be flushable, ecofriendly, and hypoallergenic to prevent infection, irritation, and toxins. Accordingly, the device 100 is all natural making it safe and conformable for women to wear, and easy to dispose of after use.

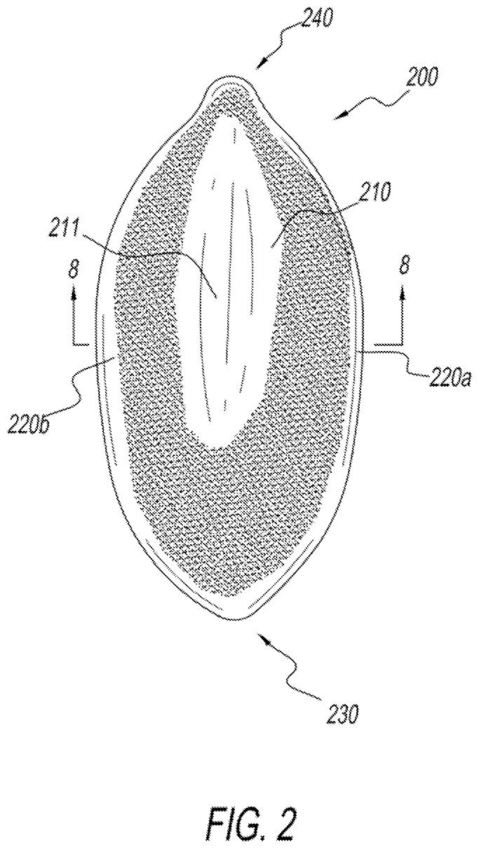

Referring now to FIG. 2, the feminine sanitary fold device 200 is depicted. FIG. 2 shows a top view of the device. Here, lateral side portions 220a and 220b are shown, extending downward from the curved portion 210 including the absorbent material 211. Also, the front fold portion 230 and the rear fold portion 240 are shown.

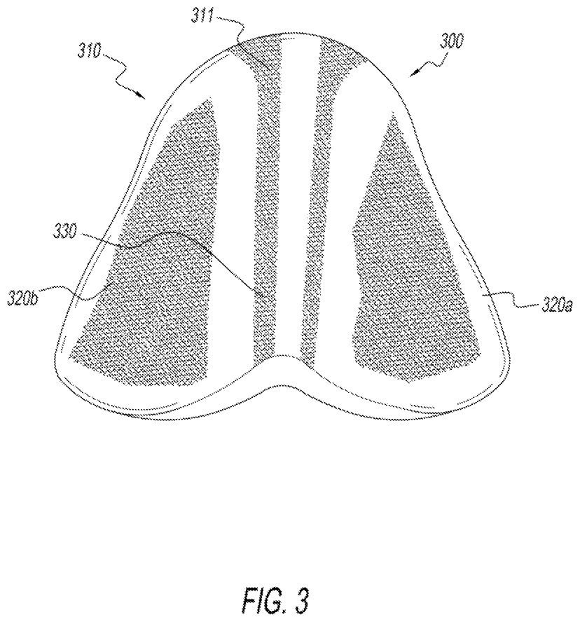

In FIG. 3, another view of the feminine sanitary fold device 300 is depicted. FIG. 3 shows a front view of the device. The front fold portion 330 is prominently shown. Also, the lateral side portions 320a and 320b are shown, and the curved portion 310 including the absorbent material 311.

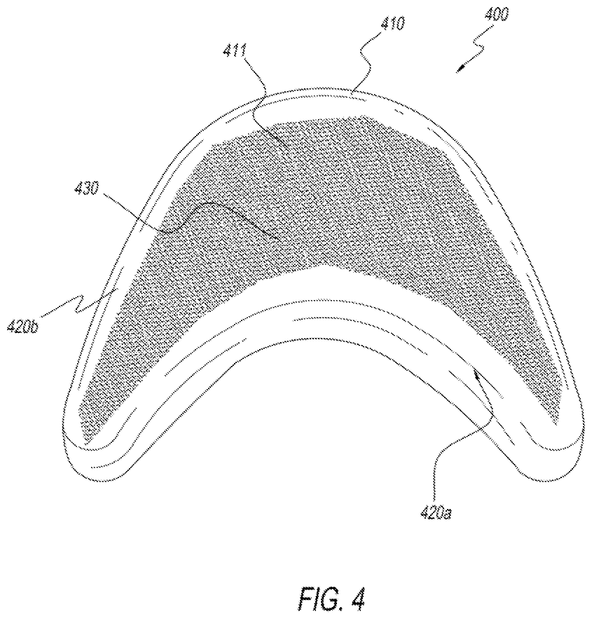

Now referring to FIG. 4, another front view of the feminine sanitary fold device 400 is depicted. The front fold portion 430 is prominently shown. Also, the lateral side portions 420a and 420b are shown, and the curved portion 410 including the absorbent material 411.

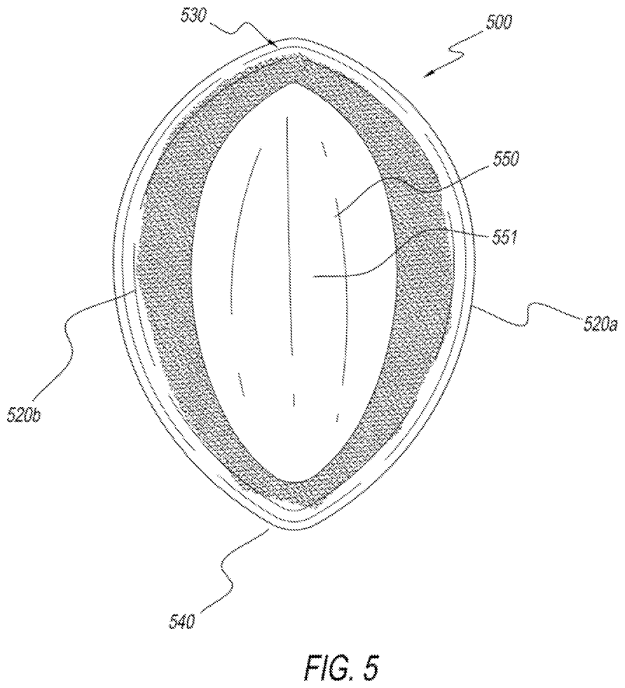

In FIG. 5, a bottom view of the feminine sanitary fold device 500 is shown. In this example, a bottom surface of device 500 is shown, illustrating the opening between the lateral side portions 520a, 520b formed by the inward bending of the curved portion 510. For example, when worn, the device 500 will have the curved portion 510 slightly inside of the vagina of the wearer, and this bottom portion exposed (e.g., not contacting the skin). The bottom surface of the device 500 can be considered the surface that is the “inside” of device 500, or the surface that is underneath of the external surface of the device 500 that is contacting the wearer's skin. In some embodiments, this bottom surface can include an additional portion of absorbent material 551.

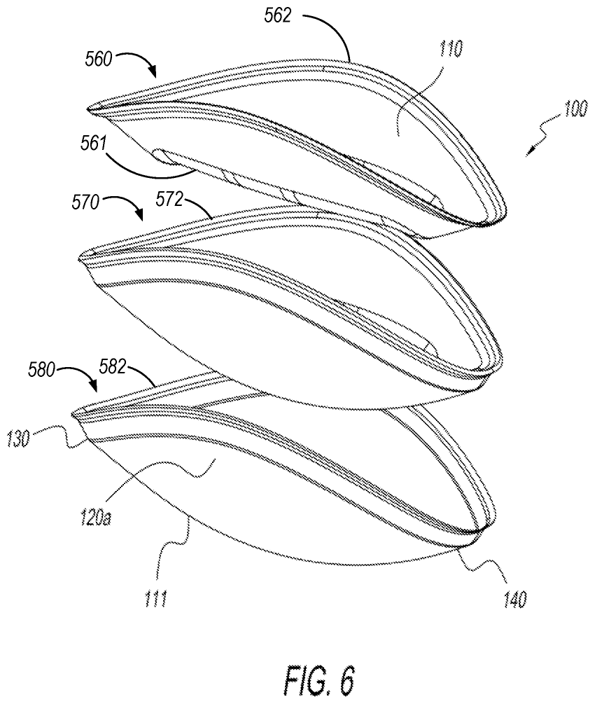

FIG. 6 depicts an exploded view of the feminine sanitary fold device 100 in order to illustrate the various layers comprising the multi-layer material. In the example of FIG. 6, the multi-layer material used to construct the feminine sanitary fold device 100 includes three principal layers, namely: a sealed non-permeable membrane layer 560; an absorbent layer 570; and a one-way membrane layer 580. According to an embodiment, the one-way membrane layer 580 forms the exterior (e.g., outer) surface of the feminine sanitary fold device 100. For instance, the one-way membrane layer 580 can be considered the top of the feminine sanitary fold device 100, as previously described in FIG. 2. Thus, the outer layer of the feminine sanitary fold device 100, it is the one-way membrane layer 580 of the multi-layer material that is closest to (and coming into contact with) the wear's skin and anatomy when the device 100 is fully inserted into the folds of the wearer's labia. Accordingly, the one-way membrane layer 580 can be designed as material that allows for bodily fluids to move away from the user and to be collected for disposal and/or flushing. For example, the one-way membrane layer 580 can be constructed as a thin layer of semi-permeable material including a porous membrane, where the pores permit liquid, such as the user's bodily fluids, to pass through the membrane and down to the lower layers of the multi-layer material. In other words, the feminine sanitary fold device 100 leverages the one-way membrane layer's 580 ability to be permeated and gravity to ensure that the bodily fluids travel in “one direction,” which is down through that exterior layer of the multi-layer material and away from the user's body. Further, due to the one-way membrane layer 580 being semi-penetrable (as opposed to fully penetrable), the one-way membrane layer 580 can also prevent liquid from traveling back up and through the membrane and undesirably contacting with the user's skin and anatomy.

As seen in FIG. 6, the sealed non-permeable membrane layer 560 can be an interior layer of the feminine sanitary fold device 100. As the innermost layer of the feminine sanitary fold device 100, the sealed non-permeable layer 560 is below the absorbent layer 570 and the one-way membrane layer 580, and is farthest away from the wear's skin and anatomy when the device 100 is fully inserted. As an example, the sealed non-permeable membrane layer 560 forms the bottom surface of the feminine sanitary fold device 100, which is also considered as the “inside” of device 100, as described above in reference to FIG. 5. The sealed non-permeable membrane layer 560 can be constructed from a non-permeable material that acts as a seal by completely preventing liquids, such as bodily fluids, from crossing through the membrane and ultimately penetrating the material. For example, when the feminine sanitary fold device 100 is in use, the user's bodily fluids will move away from the user and travel down through the upper layers (e.g., layer 580, 570) of the multi-layer material. However, is any of this fluid reaches the sealed non-permeable membrane layer 560, its non-permeable material will block the fluids from coming through this layer, causing the liquid to be held in the “inside” the device 100. Thus, the sealed non-permeable membrane layer 560 is designed to seal the interior, or underside, of the feminine sanitary fold device 100 in a manner that prevents any spilling or leakage.

In the embodiment of FIG. 6, the absorbent layer 570 forms a “middle” layer of the feminine sanitary fold device 100. Particularly, the absorbent layer 570 is in-between the one-way membrane layer 580 and the sealed non-permeable membrane layer 560. The absorbent layer 570 can be constructed from a lightweight, flexible, and absorbent material, such as organic bamboo, absorbent cotton, and the like. As a result, the absorbent layer 570 is designed to absorb a substantial amount of liquid, such as menstrual fluid, that permeates through the top surface of the feminine sanitary fold device 100, namely the one-way membrane layer 580, prior to reaching the bottom surface of the feminine sanitary fold device 100, namely the sealed non-permeable membrane layer 560.

FIG. 6 illustrates that the feminine sanitary fold device 100 can be constructed with the one-way membrane layer 580 as the exterior layer of the device 100, the absorbent layer 570 as the “middle” layer of the device 100, and the sealed non-permeable layer as the interior layer, or the “inside”, of the device 100. FIG. 6 also shows that the each of the layers include a ridge, or a lip, at the top edge the surface that extends along its perimeter. As seen in FIG. 6, the sealed non-permeable membrane layer 560 has a ridge 562, the absorbent layer 570 includes a ridge 572, and the one-way membrane layer 580 has a ridge 582. Each of the ridges 562, 572, and 582 are contoured to have the slight protrusion (or lip) insert under the folds of the labia majora and labia minora, thereby providing a better fit and firmer hold of the feminine sanitary fold device 100 into the natural contours of the user's anatomy.

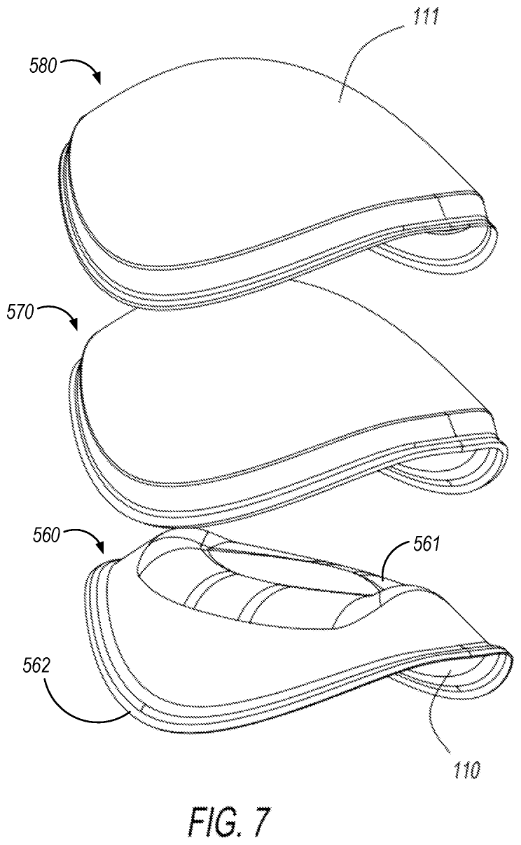

FIG. 7 depicts the layers 560, 570, and 580 of the multi-layer material for the feminine sanitary fold device 100. FIG. 7 depicts the sealed non-permeable membrane layer 560, absorbent layer 570, and one-way membrane layer 580 illustrates having a substantially similar structure and function as described in reference to FIG. 6. Thus, for purposes of brevity, the details of each of the layers 560, 570, and 580 are not described again in detail in reference to FIG. 7.

In contrast, FIG. 7 illustrates the multi-layers in an arrangement that reflects the positioning of feminine sanitary fold device 100 in use. For example, after the feminine sanitary fold device 100 is installed, the one-way membrane layer 580 may be the “top” surface of the device 100, the absorbent layer 570 can be the “middle” layer of the device 100, and the sealed non-permeable layer 580 can be the “bottom” surface of the device 100.

In addition, the perspective of the feminine sanitary fold device 100 depicted in FIG. 7 prominently shows a crease 561 that can be included in the sealed non-permeable membrane layer 560. Crease 561 is illustrated as a raised ring (e.g., substantially circular in geometry) portion on the top surface of the sealed non-permeable layer 560. For example, the crease 561 is designed at a depth of 0.75 inches from the top surface of the sealed non-permeable membrane layer 560. The crease 561 can serve as an added barrier that further supports the leakage prevention features of the feminine sanitary fold device 100. For example, the slightly elevated sections of the crease 561 can obstruct movement of any liquid that is held inside of the feminine sanitary fold device 100 by the sealed non-permeable layer 560, blocking the liquid from leaking down the sides of the fold and outside of the device 100.

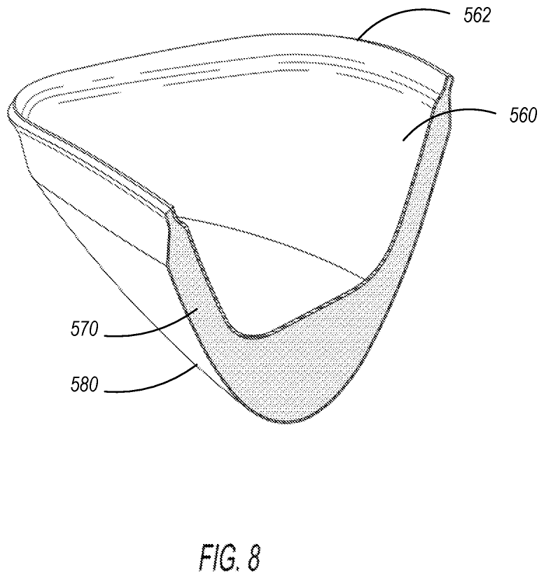

FIG. 8 depicts a cut-away view of the multi-layer material of the feminine sanitary fold device 100, including the sealed non-permeable membrane layer 560, the absorbent layer 570, and the one-way membrane layer 580. In some embodiments, a concentration on the amount of absorbent material comprising the absorbent layer 570 may be tapered. For example, the top and center area of the absorbent layer 570 can have a highest concentration of absorbent material. The concentration of absorbent material can gradually taper down from the center, towards the edge of device 100. For instance, an area of the absorbent layer 570 that is 0.5 inches away from the center of the layer 570 may have a higher concentration of absorbent material that an area that is 0.75 inches from the center; and the area that is 0.75 inches away from the center of the layer 570 may have a higher concentration of absorbent material that an area that is at the very outer edge of the device 100 (proximate to ridge 562). In most cases, the section of the absorbent layer 570 near the outer edge of the device 100 has the lowest concentration of absorbent material of the layer 570.

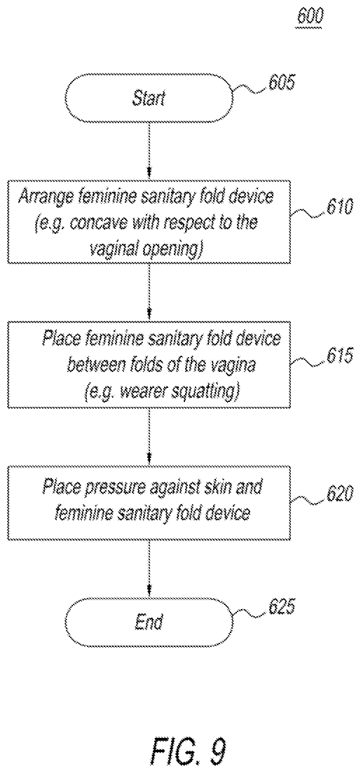

Now referring to FIG. 9, an example of a method 600 for installing the feminine sanitary fold device (shown in FIG. 1-FIG. 8) is shown. The method 600 can start at 605. Next, at operation 610, the user, namely a woman intending to wear the feminine sanitary fold device, can arrange the feminine sanitary fold device such that the “fold” is positioned towards the vaginal opening. In other words, the feminine sanitary fold device is positioned such that the curve, or bend, of the “fold” is concave with respect to the inward curve of the vagina (or the vaginal opening), and the top portion of the device (e.g., exterior surface including the one-way membrane layer shown in FIG. 7) is facing upwards in the direction of the vaginal opening. For example, the user can hold the feminine sanitary fold device parallel to the vagina area in a lengthwise manner, such that the length of the device runs parallel to the length of the vagina.

Next, at operation 620, the feminine sanitary fold device can be placed slightly placed inside of the vaginal area, resting between the labia majora and labia minora. In order to place the device inside of the vagina, the wearer can put their body in a position that generally opens the folds of the vagina. For example, the wearer can squat, which separates the labia majora and labia minora, creating a space where the feminine sanitary fold device can be placed. As previously described, the feminine sanitary fold device has dimensions (e.g., length and width) and flexibility that allow if to be form-fitting to the shape of vagina, and ultimately held securely in place without adhesive. Further, the ridge (or lip) of the feminine sanitary fold can rest in the fold of the labia majora and labia minora. Also, the feminine sanitary fold device is placed to allow the curved portion, and the absorbent material, to be slightly in contact with the vaginal opening to collect any fluid that is discharged. It is important to note that feminine sanitary fold device can be manually positioned and placed by the wearer. Further, the feminine sanitary fold device can be placed inside of the vaginal area without using plastic applicator, which is common to tampons, which can cause irritation and discomfort.

Thereafter, once the feminine sanitary fold device is placed inside of the vagina, the wearer can place their body into a position that causes the labia majora and labia minora to apply firm pressure against the device at operation 620. Thus, operation 620 causes the feminine sanitary fold device to be held into place, after initially being positioned inside of the vagina. For example, the wearer can stand up out of the squatting position in order to generally close their legs, and closing the folds of the vagina. As mentioned above, the pressure from being pressed between the labia majora and labia minora (as the wearer stands with their legs closed) holds the feminine sanitary fold device in stable and secure position inside of the vagina. Thus, the feminine sanitary fold device is secured in a comfortable position during use, without having to use adhesives or being inserted deeply inside of the vagina. With this stability, the potential of unintended shifting and leaking is greatly reduced by using the feminine sanitary fold device. The method 600 can end at operation 625.

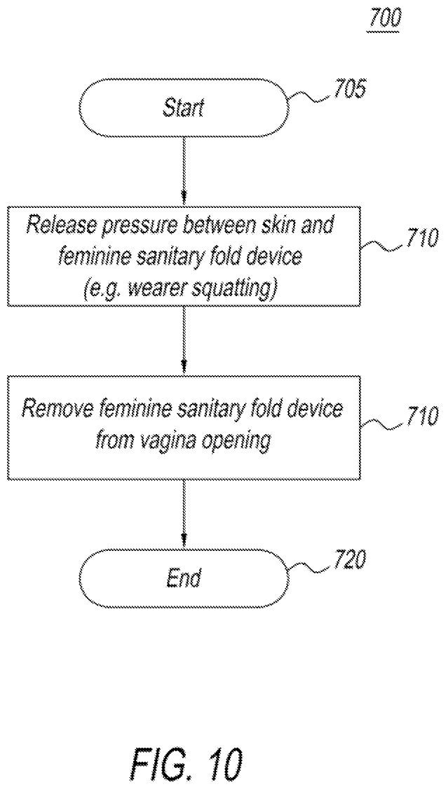

Now referring to FIG. 10, a method 700 for removing the feminine sanitary fold device is shown. The operation can start at operation 705. Next, at operation 710, the wearer can put their body into a position that releases pressure between the folds of the vagina and the device. As alluded to above, the wearer can squat down, which typically causes separation of the labia majora and labia minora folds and the vagina to open. This cases the feminine sanitary fold device to be loosened, or not as firmly gripped between the vaginal folds.

Subsequently, at operation 715, the feminine sanitary fold device can be removed from its position inside of the vagina. Removing the feminine sanitary fold device should be easy due to the pressure being loosened. For instance, the wearer should be able to place a finger into the opening of the “fold” that is not in contact with the skin (e.g., bottom surface of the device) and gently pull downward to remove the device from the vagina.

In some cases, operation 715 can involve disposing of the feminine sanitary fold device. As an example, because the feminine sanitary fold device is constructed from flushable, biodegradable material, the wearer can simply place the device into a toilet. Accordingly, the device can be dispose of in a sanitary and simple manner, after removal in method 700. Next, the method 700 can end at operation 720.

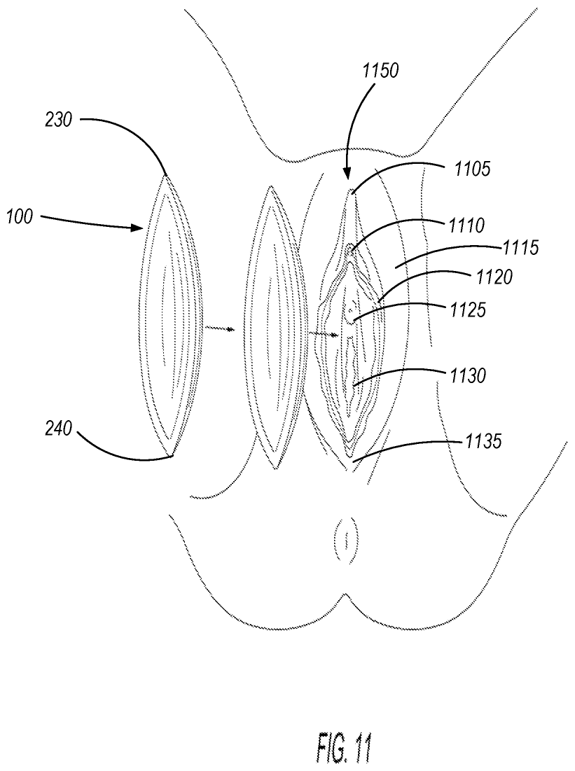

FIG. 11 depicts an example of the feminine sanitary fold device 100 that is arranged in a position to be installed by a wearer. In particular, FIG. 11 illustrates an example of a vaginal area 1150 (commonly referred to as the vulva) which is an area of a female body that includes the external parts of the female genitalia. As shown in FIG. 11, a vaginal area 1150 can typically include, but is not limited to: a prepuce 1105; a clitoris 1110; a labia majora 1115; a labia minora 1120; a urethral opening 1125; a vaginal opening 1130; and a fourchette 1135. Also, as seen in FIG. 11, the vaginal area 1150 is slightly opened, creating some spacing between the opposite folds of the labia majora 1115 and the labia minora 1120. For example, this opening of the vaginal area 1150 can be formed when a user is in a squatting position in order to install the feminine sanitary fold device 100.

As seen, the feminine sanitary fold device 100 may be positioned such that the length of the device 100 is aligned vertically parallel to the shape of the vaginal area 1150. For example, a left lateral side portion of the device 100 may be aligned with folds of the left side of the labia majora 1115 and labia minora 1120, and a right lateral side portion of the device 100 may be aligned with folds of the right side of the labia majora 1115 and labia minora 1120. Further, the front fold portion 230 of the device 100 is arranged to be similarly proximate to the front of the vaginal area 1150, shown as facing the prepuce 1105 in the installation position. The rear fold portion 240 of the device 100 is proximate to the rear of the vaginal area 1150, shown as facing the fourchette in the installation position.

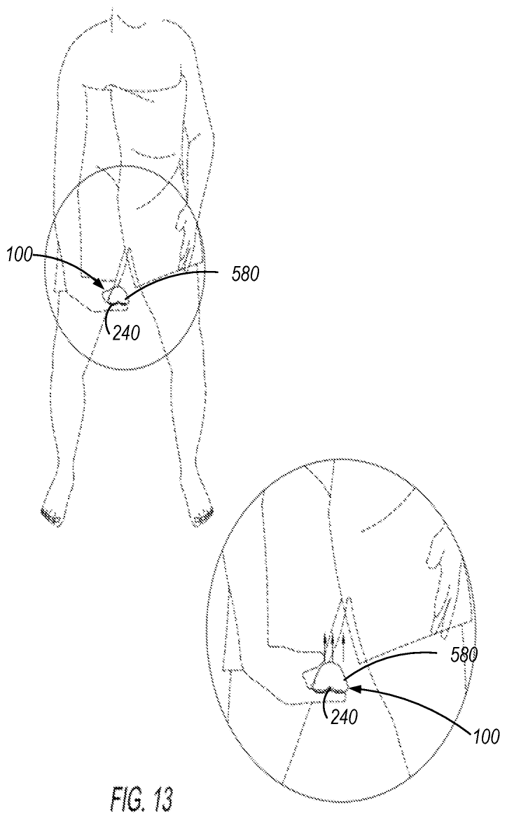

FIG. 11 also depicts that the feminine sanitary fold device 100 can initially be held at a distance away from the vaginal area 1150, for instance 1 inch-6 inches away from the vaginal area 1150, as the user arranges the device 100 into the proper installation position. As an example, FIG. 13 depicts a user standing, as the device 100 is being held in one hand. The user can hold the device 100 generally away from the vaginal area as they begin to get the device 100 situated, and can move the device 100 closer to the body in order to properly arrange the device 100 into the initial position (being parallel to the labial folds) to be installed. In the close-up view of FIG. 13, the device 100 is shown to have the top surface, including the one-way membrane layer 580 of its multi-layer material, facing towards the opening of the vaginal area when it is situated in the correct position to be installed.

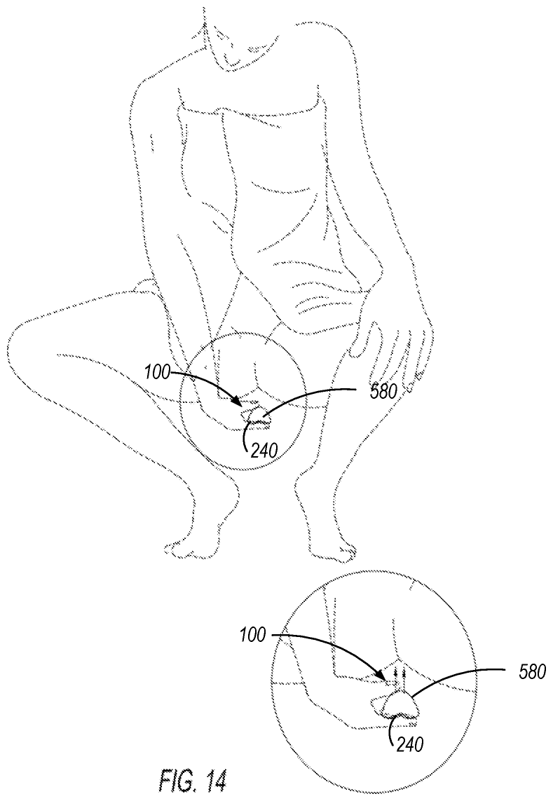

After the feminine sanitary fold device 100 is arranged in this initial installation position, the user can then move the feminine sanitary fold device 100 into the opening of the vaginal area 1150 in order to be inserted between this spacing of the labial folds. For instance, the user can use one hand to push the feminine sanitary fold device 100 such that the device 100 moves in the direction towards the vaginal area 1150 (indicated by arrow) to ultimately insert the device 100 into the opening between the folds of the labia majora 1115 and labia minora 1120. This is also depicted in FIG. 14. Particularly in FIG. 14, the user is shown in the squatting position, which opens the labial folds of the vaginal area. Further, the user is holding the device 100 in their hand such that the device 100 is parallel, aligned lengthwise, to the vaginal area, which allows the device 100 to be easily be inserted into the opening of the labial folds. In the close-up view of FIG. 14, the user is shown pushing the device 100 in an upward direction (indicated by arrow) towards the vaginal area in order to be ultimately placed into the folds of the labia. The device 100 is held in the position having its top surface, including the one-way membrane layer 580 of its multi-layer material, facing towards the opening of the vaginal area as it is being inserted.

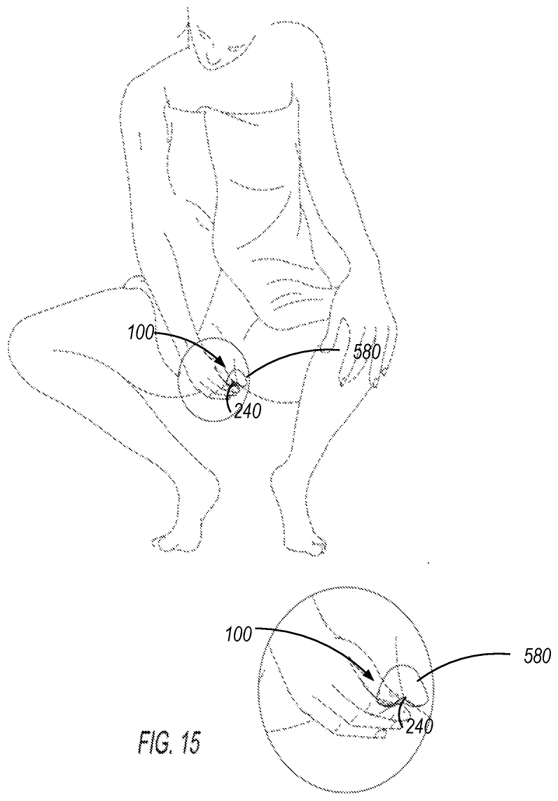

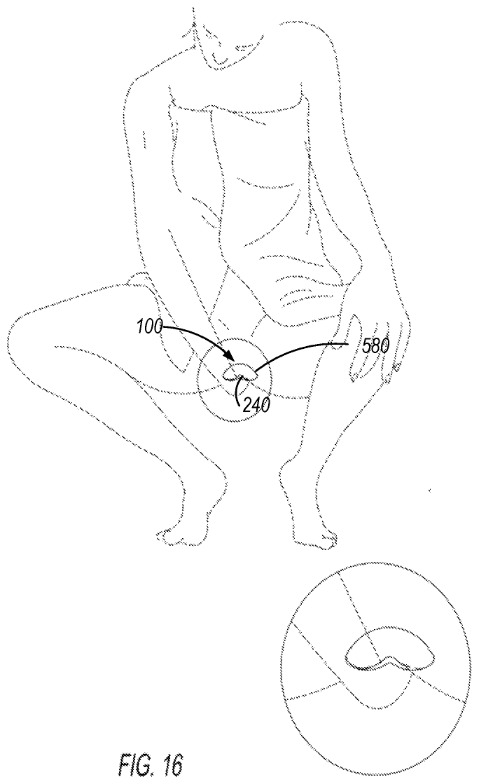

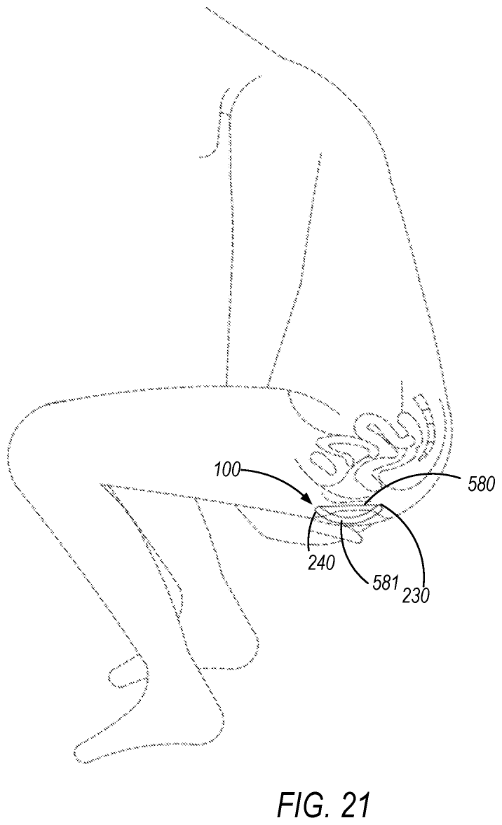

Referring back to FIG. 11, the user can continue to push the device 100 until the lateral side portions of the device's 100 folds rest comfortably and securely in the labial folds, referred to herein as the fully inserted position. An example of this part of the installation is shown in FIG. 15-FIG. 16. That is, FIG. 15 shows the user, still in the squatting position, pushing the device 100 as it is beginning insertion into the labial folds. FIG. 16 shows the user having the device 100 fully inserted into the labial folds. Additionally, FIG. 21 illustrates a side view of the device 100 in the fully inserted position, inside of the labial folds of the user.

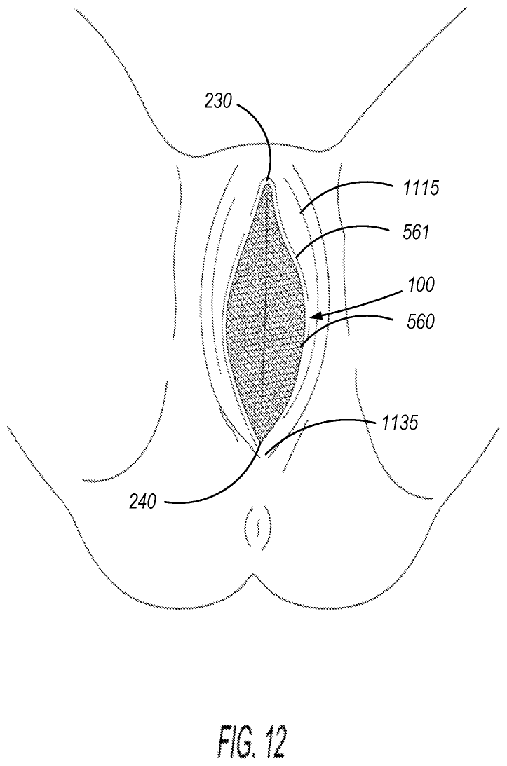

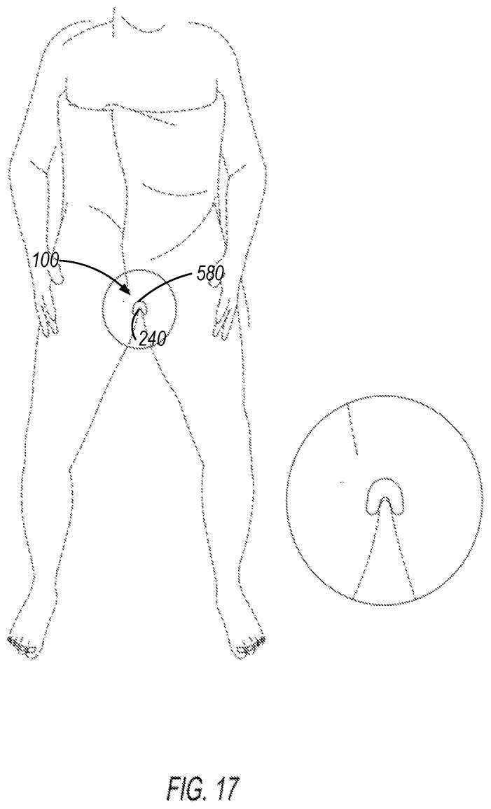

FIG. 12 depicts an example of the feminine sanitary fold device 100 while in the fully inserted position, for instance after being installed into the labial folds of the user's vaginal area 1150. For example, in the fully inserted position, the ridged edges (or lips) of the device 100 may be slightly pressing against the labia folds of the user's vaginal area 1150. FIG. 12 illustrates a bottom view of the device 100 with respect to the vaginal area 1150. Thus, as seen, the bottom surface of the feminine sanitary fold device 100 remains exposed (e.g., outside of opening of the vaginal area 1150), since the device 100 does not have to be inserted into the vagina in the same manner as many existing feminine products. After the device 100 is in the fully inserted position, the user can stand up, which creates pressure such that the lateral side portions of the device's 100 folds contract, coming closer together and pressing more firmly into the labial folds of the user's vaginal area. Thus, locking the device 100 securely into the fully inserted position, even as the user stands, moves, and performs normal tasks throughout the day. For instance, FIG. 17 illustrates the user standing, while her labial folds contract folds of the device 100, locking the device 100 stably into place.

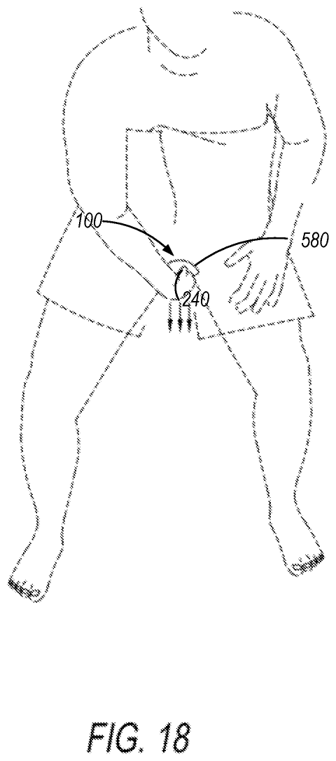

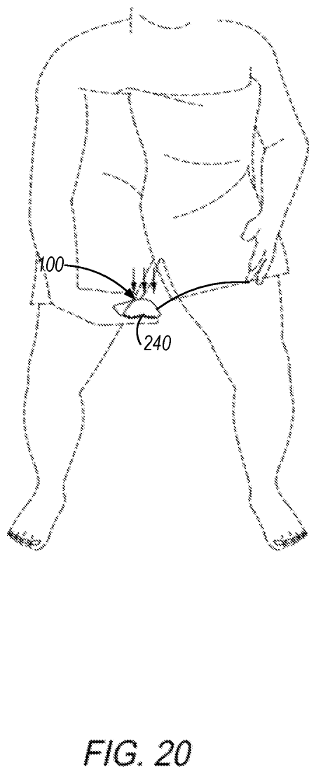

In FIG. 18 an example of a user removing the feminine sanitary fold device 100, for instance to discard after use, is shown. The user is shown in a slightly squatting position, which releases pressure between the labial folds and the device. In FIG. 20, the user is shown completely squatting down, which typically causes separation of the labia majora and labia minora folds, and generally opening the vaginal area. This causes the feminine sanitary fold device 100 to be loosened, or not as firmly gripped between the vaginal folds. Particularly, the user can take a hand to easily remove the feminine sanitary fold device 100 from between the labial folds due to the pressure being loosened. For instance, the user can to place a finger into the opening of the “fold” that is not in contact with the skin (e.g., bottom surface of the device), and down to remove the device 100 from being inserted into the vaginal area.

Subsequently, as shown in FIG. 20, the feminine sanitary fold device 100 can be completely removed from its position inside of the vaginal area, being outside and/or away from the vaginal area. In the fully removed position, the device 100 can be disposed of.

Additional aspects of the disclosure set forth in part in the description above, and in part will be obvious from the description, or can be learned by practice of the disclosure. The advantages of the disclosure will be realized and attained by means of the elements and combinations particularly pointed out in the appended claims. It is to be understood that both the foregoing general description and the detailed description are and explanatory only and are not restrictive of the disclosure, as claimed. Both quantitative and qualitative assessments and feedback also provide support for the novelty and non-obvious nature of this disclosure. The disclosure overcomes the shortcomings of conventional cups, pads, tampons, and conventional sanitary devices.

In one or more aspects, the disclosure may comprise shape conforming materials the shape conforming materials further comprising at least one of: a liquid permeable membrane and/or substrate material, a liquid impermeable membrane and/or substrate material configured as a baffle, a fluid absorbent membrane and/or substrate material, a physiologically hydrous membrane and/or substrate material, an antimicrobial membrane and/or substrate material, a transfer layer membrane and/or substrate material, a moisture wicking membrane and/or substrate material, a hypoallergenic fiber material including but not limited to: silk, cotton, sheepskin, linen, flax, and the likeness thereof. In one or more instances, these aforementioned membranes and/or substrates may further comprise synthetic materials, organic natural materials, or any combination of synthetic and organic natural materials including but not limited to: biodegradable resin, polylactic acid, spun bond nonwoven fabric, acetate, synthetic fiber, laminated continuous fibers, high polymer absorbing material, carboxymethylcellulose fiber, carboxyethylcellulose fiber, laminated paper, laminated film, laminated tissue, polybutylene succinate, silicone, starch, polyvinylalcohol, low-density polyethylene, and the likeness thereof. In one or more instances these one or more layers may be connected by various conventional and non conventional adhering methods including but not limited to bonding by water soluble and/or water expanding adhesives, polyvinylalcohol and the likeness thereof, a heat seal, a hydrogen bond, and the likeness thereof.

In one or more instances, the disclosure may comprise one or more self-cleaning and self-sanitizing coatings including but not limited to fabrics and textile materials pre-treated by impregnation or incorporation with antimicrobial pesticides for protection purposes against bacteria and fungi that are pathogenic for humans and animals. The one or more self-cleaning and self-sanitizing coatings comprising materials including but not limited to: self-cleaning and self-sanitizing coatings on plastic fabrics, incorporating active inorganic matter alone (i.e., photo-catalytic TiO2 anatase and Ag(+) ions) inside an organic inorganic hybrid binder which when applied onto coextruded polyvinylchloride-polyester fabrics by air-mix spraying and other known methods to one of ordinary skill in the art drying at ambient and other temperatures then provides for resulting coatings were characterized for their self-cleaning and self-sanitizing ability according to standardized testing procedure and/or applicable international regulation. In one or more instances, the disclosure may comprise at least one antimicrobial textile product and/or eco-friendly, renewable resource, biodegradable material.

In one or more instances, the present disclosure comprises a biodegradable material and/or a water dispersible material and/or a water-soluble material. After using the disclosed device comprising one or more the aforementioned materials, it can be disposed into a toilet to flush, thereby the destruction of the disclosure may be easily and sanitarily achieved and the garbage in a toilet can be decreased.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A feminine sanitary fold device, comprising:
a body having a curved portion, wherein the curved portion is contoured to the female genitalia; and
two lateral side portions that extend downward from the curved portion forming a fold, wherein the fold is contoured to insert between the labial folds of the female genitalia; and
a top surface of the body comprising an absorbent material to absorb bodily fluid secreted from the female genitalia.

2. The feminine sanitary fold device of claim 1, wherein the body comprises dimensions to be form-fitting to the shape of the female genitalia.

3. The feminine sanitary fold device of claim 2, wherein the dimensions comprise a length to be parallel aligned to a length of the female genitalia.

4. The feminine sanitary fold device of claim 2, wherein the dimensions comprise a width to be aligned to a width of the female genitalia.

5. The feminine sanitary fold device of claim 1, wherein the absorbent material comprises at least one of: organic material, organic bamboo, and absorbent cotton.

6. The feminine sanitary fold device of claim 1, wherein the top surface comprises a one-way membrane layer.

7. The feminine sanitary fold device of claim 6, wherein the one-way membrane layer comprises a semi-permeable material that permeates the bodily fluid through the layer.

8. The feminine sanitary fold device of claim 7, wherein the one-way membrane layer comprises a semi-permeable material that permeates the bodily fluid through the one-way membrane layer.

9. The feminine sanitary fold device of claim 8, further comprising a bottom surface of the body for collecting the bodily fluid secreted from the female genitalia.

10. The feminine sanitary fold device of claim 9, wherein the bottom surface comprises a sealed non-permeable membrane layer that block the bodily fluid from penetrating through the sealed non-permeable membrane layer.

11. The feminine sanitary fold device of claim 10, comprising an absorbent layer between the bottom surface disposed between the bottom surface and the top surface.

12. The feminine sanitary fold device of claim 11, wherein the absorbent layer comprises an absorbent material.

13. The feminine sanitary fold device of claim 12, wherein the absorbent layer comprises various concentrations of the absorbent material.

14. The feminine sanitary fold device of claim 12, wherein the various concentrations of the absorbent material are tapered from a center of the body to an edge of the fold.

15. The feminine sanitary fold device of claim 1, comprising a ridge along an edge of the fold for inserting between the labial folds of the female genitalia.

16. A method, comprising:
arranging a feminine sanitary fold device into an installation position;
placing the feminine sanitary fold device in an opening between labial folds of a vagina into an inserted position; and
placing pressure against skin of the vagina and the feminine sanitary fold device to secure the feminine sanitary fold device into the inserted position.

17. The method of claim 16, wherein placing the feminine sanitary fold device in an opening between the labial folds of the vagina comprises a user squatting and placing a lip of the feminine sanitary fold device between a labia minora and a labia majora of the vagina to move the feminine sanitary fold device into the inserted position.

18. The method of claim 16, wherein placing pressure against the skin and the feminine sanitary fold device comprises a user standing and contracting folds of the feminine sanitary fold device together.

19. The method of claim 16, wherein the installation position comprises aligning the feminine sanitary fold device parallel to the labial folds of the vagina and aligning the feminine sanitary fold device concave with respect to the opening between labial folds of the vagina.

20. A method, comprising:
releasing pressure between skin of a vagina and a feminine sanitary fold device in an inserted position between labial folds of the vagina; and
removing the feminine sanitary fold device from an opening between the labial folds of the vagina.
